(19) United States
(12) Patent Application Publication
(10) Pub. No.: US 2026/0283588 A1
(43) Pub. Date: Sep. 24, 2026

(54) DETECTING OBJECTS BY VIBRATION-ELICITED SHIMMERING WITH A VIBRATION-DEDICATED ULTRASOUND APPLICATION

(71) Applicant: Mayo Foundation for Medical Education and Research, Rochester, MN (US)

(72) Inventors: Christine U. Lee, Byron, MN (US); Dan Moldoveanu, Rochester, MN (US); Matthew W. Urban, Rochester, MN (US); James F. Greenleaf, Rochester, MN (US); Christopher L. Deufel, Rochester, MN (US); Eric E. Brost, Eagan, MN (US); Bradley J. Stish, Rochester, MN (US); Luiz D. Vasconcelos, St. Paul, MN (US)

(21) Appl. No.: 19/479,122

(22) PCT Filed: Apr. 29, 2024
(86) PCT No.: PCT/US2024/026860

§ 371 Date: Oct. 27, 2025

Related U.S. Application Data
(60) Provisional application No. 63/498,778, filed on Apr. 27, 2023.

(51) Int. Cl. A61B 8/08; A61B 8/00; A61B 90/00

(52) U.S. Cl. CPC A61B 8/0825; A61B 8/085; A61B 8/488; A61B 90/39; A61B 2090/3925

(57) ABSTRACT

A vibrating device is used to improve the visualization of implanted biopsy markers, or other objects, by ultrasound. Externally applied mechanical vibrations propagate in the subject's tissue where they interact with the biopsy marker and cause it to vibrate. Ultrasound data acquired while the biopsy marker is vibrating include a Doppler ultrasound signature that can be analyzed to determine the location of the biopsy marker.

### CROSS-REFERENCE TO RELATED APPLICATIONS

This application claims the benefit of U.S. Provisional Patent Application Ser. No. 63/498,778, filed on Apr. 27, 2023, and entitled “DETECTING OBJECTS BY VIBRATION-ELICITED SHIMMERING WITH A VIBRATION-DEDICATED ULTRASOUND APPLICATION,” which is herein incorporated by reference in its entirety.

### BACKGROUND

When a breast mass or lymph node is biopsied, a small marker or clip is usually left behind in order to more easily identify the region of interest. These markers are usually readily visualized by mammography, but can be difficult to find by ultrasound. For this reason, surgeons usually resort to implantable radioactive “seeds” to be placed near a biopsy marker, and which can be detected with the Geiger counter at the time of surgery to help guide the surgeon to the area of interest (usually a cancer).

If the initially placed marker could be easily identified by ultrasound, surgeons could omit placing the radioactive seed and use the biopsy marker as a guide to identify the tumor under ultrasound guidance. Unfortunately, most biopsy markers are small and difficult to identify by B-mode ultrasound, hence the necessity of an additional, invasive localization procedure.

### SUMMARY OF THE DISCLOSURE

It is an aspect of the present disclosure to provide a method for determining a location of an object in a region-of-interest in a subject using an ultrasound system. Mechanical vibrations are generated in the region-of-interest containing the object using an external vibrational device. Ultrasound data are acquired from the region-of-interest using an ultrasound system while the mechanical vibrations are propagating within the region-of-interest, such that the object vibrates in response to the propagating mechanical vibrations. The ultrasound data are processed with a computer system to determine a location of the object within the region-of-interest based on Doppler ultrasound signatures generated by vibrations of the object interacting with ultrasound waves incident on the object. The location of the object may then be presented to a user via the computer system.

### BRIEF DESCRIPTION OF THE DRAWINGS

FIG. 1 illustrates an example of using an external vibrational device to generate mechanical vibrations to propagate in a region-of-interest to vibrate an object contained therein, and to acquire ultrasound data from the region-of-interest containing the vibrating object.

FIG. 2 is a flowchart setting forth the steps of an example method for visualizing or otherwise determining the location of an object (e.g., a biopsy marker) in a region-of-interest by simultaneously vibrating the object via an external vibration device coupled to the skin surface of the subject and acquiring ultrasound data in response thereto.

FIG. 3 illustrates the use of white noise as a signal input to a drive a signal generator for generating mechanical vibrations with an external vibration device.

FIG. 4 illustrates images generated from ultrasound data acquired using the methods described in the present disclosure, which depict the locations of two objects contained within the imaged region-of-interest.

FIGS. 5A and 5B illustrate example images showing a positive shimmering signature from a biopsy marker (FIG. 5A) and a negative shimmering signature from a biopsy marker (FIG. 5B); that is, where the tissue environment surrounding the biopsy marker creates a positive shimmering signature, but the biopsy marker is non-shimmering.

FIG. 6 is a block diagram of an example system for detection or otherwise localizing an object using an ultrasound system.

FIG. 7 is a block diagram of example components that can implement the system of FIG. 6.

FIG. 8 is a block diagram of an example ultrasound system that can implement the methods described in the present disclosure.

### DETAILED DESCRIPTION

Described here are systems and methods for improving the visualization of implanted object, such as implanted biopsy markers, using ultrasound. An external vibrational device is used to introduce mechanical vibrations in the tissue of a subject, such that the mechanical vibrations propagate through the subject's tissue and interact with the implanted object. The region-of-interest containing the object is then imaged using ultrasound (e.g., Doppler ultrasound) and the images analyzed to determine the location of, or otherwise visualize, the object. The ultrasound waves incident on the object interact with the vibrating surface of the object (caused by the externally applied mechanical vibrations) and generate a unique Doppler ultrasound signature in response thereto. By identifying the Doppler ultrasound signature in the acquired ultrasound data, the location of the object can be accurately determined.

As a non-limiting example application, the disclosed systems and methods can advantageously improve the visualization of biopsy markers used in breast surgery and axillary (i.e., armpit) surgery. When a breast mass or lymph node is biopsied, a small marker is usually left behind in order to more easily identify the region-of-interest. These markers may be visualized by mammography, but can be difficult to find by ultrasound. Accordingly, surgeons usually resort to implantable radioactive seeds placed near the biopsy marker, which can be detected with a Geiger counter at the time of surgery, and which can help guide the surgeon to the region-of-interest (usually containing a cancer).

It is an advantage of the disclosed systems and methods that biopsy markers can be used instead of radioactive seeds and accurately visualized using ultrasound. As a result, surgeons could omit placing a radioactive seed and instead use the biopsy marker as a guide to identify the tumor under ultrasound guidance. Although most biopsy markers are small and difficult to identify by B-mode ultrasound, the systems and methods described in the present disclosure can overcome this challenge and provide improved visualization of the biopsy marker. Advantageously, this technique can allow a surgeon to avoid the placement of a radioactive seed, sparing the patient an additional procedure, reducing their exposure to radiation, and further expediting their surgical management.

As described above, by placing a vibrational device (e.g., a mechanical actuator, a mechanical shaker) in proximity to the biopsy marker (e.g., on the skin surface near the biopsy marker), an ultrasound signal may be evoked in the biopsy marker, making it more visible on Doppler ultrasound such as color-flow mode Doppler ultrasound.

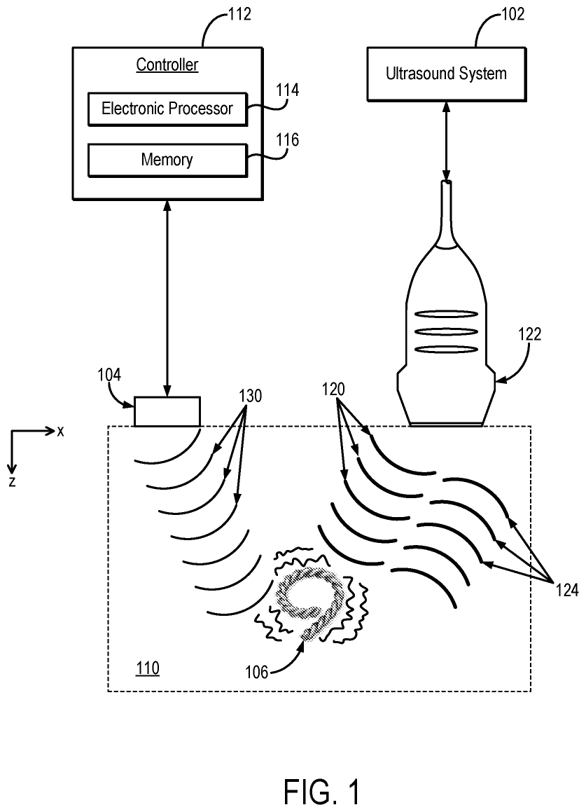

By way of example, FIG. 1 illustrates an example configuration of using an ultrasound system 102 and external vibrational device 104 to determine the location or otherwise visualize an object 106 implanted in a region-of-interest 108 of a subject. The external vibrational device 104 may be coupled to or detached from the transducer 122.

The vibrational device 104 may include one or more mechanical actuators, mechanical shakers, or the like. The vibrational device 104 may be physically separate from the transducer 122, as illustrated in FIG. 1, or may alternatively be coupled to the transducer 122. Additionally or alternatively, more than one vibrational device 104 (e.g., more than one mechanical actuator) may be used, with one or more vibrational devices 104 coupled to the transducer 122 and one or more vibrational devices 104 being physically separate from the transducer 122. In one example, the vibrational device 104 may include a plurality of actuators (e.g., a plurality of mechanical actuators). For instance, the vibrational device 104 may include a ring or other array of mechanical actuators that may be coupled to the transducer 122, or may be physically separate from the transducer 122.

In response to a vibration stimulus, the vibrational device 104 begins to vibrate and generate mechanical vibrations 130 that propagate through the region-of-interest 108 and interact with the object 106. Ultrasound waves 120 are transmitted by a transducer 122 of the ultrasound system 102. The ultrasound waves 120 incident on the object 106 interact with the vibrating surface and/or volume of the object 106, which emits reflected ultrasound waves 124 in response thereto. The reflected ultrasound waves 124 are received by the transducer 122, or another transducer of the ultrasound system 102, and recorded as ultrasound data. As an example, the ultrasound system 102 may be operated in a Doppler imaging mode such that the ultrasound data are representative of Doppler ultrasound signals. The Doppler imaging mode may include a color Doppler imaging mode, a power Doppler imaging mode, or any other suitable mathematical analog or derived signal similar to color Doppler and/or power Doppler.

An electronic controller 112 having an electronic processor 114 and a memory 116 may be used to control the operation of the vibrational device 104. For example, the electronic controller 112 may store vibration stimulus parameter settings in the memory 116. The electronic processor 114 may retrieve the vibration stimulus parameter settings from the memory 116 and use those parameter settings to control the amplitude, frequency, vibration onsets, vibration durations, and other characteristics of the mechanical vibrations generated by the vibrational device. In some examples, the vibrational device 104 may incorporate a pressure gauge to maintain a certain pressure of the vibrational device 104 against subject. In these instances, there may be a feedback loop between applied pressure and the pressure gauge such that an optimal pressure can be applied and maintained. For example, the controller 112 can receive pressure measurements from the pressure gauge can processes the pressure measurements to provide feedback for maintaining an optimal pressure against the subject. The applied pressure to the vibrational device 104 could be in the form of manual or pneumatic pressure or added weights to the vibrational device 104.

The ultrasound system 102 may be controlled by the electronic controller 112, or by a controller and/or processor integrated with the ultrasound system 102 as described below in more detail.

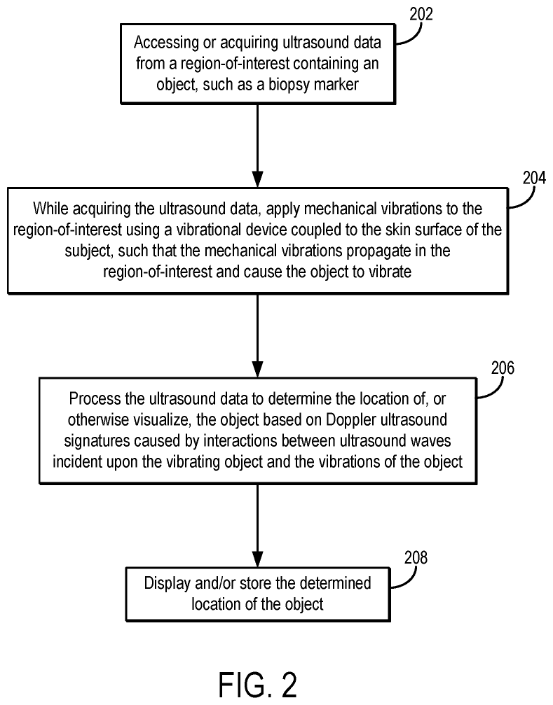

FIG. 2 illustrates a flowchart setting forth the steps of an example method for detecting and visualizing the location of an object (e.g., a biopsy marker) using an ultrasound system.

The method includes accessing ultrasound data with a computer system, as indicated at step 202. The ultrasound data are, or have been, acquired from a region-of-interest in a subject while mechanical vibrations are being applied to the subject by an external vibrational device. Accessing the ultrasound data may include retrieving previously acquired ultrasound data from a memory or other data storage medium or device. Additionally or alternatively, accessing the ultrasound data may include acquiring the ultrasound data using an ultrasound system and sending or otherwise communicating the ultrasound data to the computer system, which may be a part of the ultrasound system.

In some examples, the ultrasound data may be acquired using an acquisition technique that is optimized to visualize the vibrating biopsy marker. Additionally or alternatively, the mechanical vibrations applied by the external vibrational device may be modulated to improve the visualization of the object in the ultrasound data. For example, the vibration stimulus may be optimized to highlight the interface between the object (e.g., biopsy marker) and tissue. As an example, the vibration stimulus can be optimized by using an accelerometer or vibrometer to study the frequency and amplitude output of the vibrational device in tissue across a broad range of frequencies. Ultrasound data can be acquired and the vibration frequency/amplitude combination that best highlights the interface between an object and tissue can be selected and stored by the computer system as optimized vibration stimulus settings.

Similarly, the ultrasound detection mode can be optimized to improve visualization of the object. For instance, based on the experimental data used to assess the optimal settings for the vibration stimulus, the detection settings may also be adjusted using a band pass filter specific for the optimized frequency. Additionally or alternatively, adjustments of the Doppler transmit ultrasound frequency, Doppler pulse repetition frequency (as an effect of the Doppler velocity scale), wall filter, and the Doppler gain may be adjusted to optimally detect the vibrating object. The optimized detection mode settings can be stored by the computer system and/or the ultrasound system. For instance, the detection mode settings can be used to develop an application that can be stored and implemented by the ultrasound system.

As noted above, while the ultrasound data are acquired, mechanical vibrations are applied to the region-of-interest containing the object, as indicated at step 204. The mechanical vibrations then propagate through the region-of-interest and interact with the object causing it to vibrate. Accordingly, when the incident ultrasound waves interact with the vibrating object a Doppler ultrasound signature will be generated, as described below in more detail. In some instances, the mechanical vibrations may also interact with microbubbles in the region-of-interest, such as a Doppler shimmering signature is generated in response thereto. In some examples, the mechanical vibrations may interact with the object synergistically to enhance the visualization of the object. For instance, the object may be coated with a coating that generates enhanced Doppler signatures. As another example, the mechanical vibrations may have a frequency that is matched to a resonant frequency of the object.

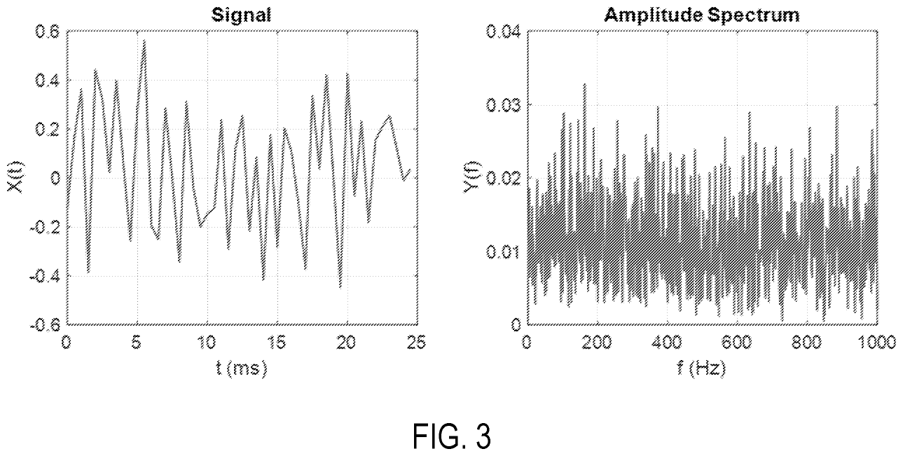

The mechanical vibrations may be applied using an external vibrational device, such as a mechanical actuator, mechanical shaker, or the like. The vibrational device is positioned on the subject's skin near the region-of-interest, may be coupled to an ultrasound transducer used to acquired ultrasound data, or the like. The vibrational device is operated to generate mechanical vibrations that propagate in the region-of-interest where they are incident upon the object. The vibrational device may be driven to generate the mechanical vibrations using a signal generator. The signal generator may drive the vibrational device at a single vibration frequency, a band of vibration frequencies (e.g., a vibration frequency band), or using white noise as an input for the signal generator (e.g., such that frequencies and/or amplitudes of the mechanical vibrations are selected based on a white noise input, as illustrated in FIG. 3). Advantageously, using white noise as the input signal to drive generation of the mechanical vibrations can result in a stronger, more reliable Doppler ultrasound signature for localizing the biopsy marker. The mechanical vibrations then cause the object to vibrate, such as by vibrating a surface of the object, a volume of the object, or so on.

As another example, the mechanical vibrations may be applied to the region-of-interest by the object itself. That is, the mechanical vibrations may be inherent to the object. As one example, the object may be controllable to cause mechanical vibrations (e.g., remotely controllable, such as via wireless communication). The entire object may be caused to vibrate, or only a portion of the object. For instance, the object may be a biopsy marker having coupled thereto an actuator that when operated caused the biopsy marker to vibrate. As another example, the object may be a surgical instrument (a catheter, needle, or the like) having an actuator or other vibrator coupled thereto.

The ultrasound data are then processed with a computer system to determine a location of the object (e.g., biopsy marker) within the region-of-interest based on Doppler ultrasound signatures generated by vibrating the object, as indicated at step 206. For instance, the interaction of ultrasound waves incident on the object and the vibrating surface and/or volume of that object causes a Doppler ultrasound signature that can be measured or otherwise analyzed to determine the location of the object. In some examples, processing the Doppler ultrasound data may include overlaying the Doppler ultrasound data on B-mode images also acquired from the region-of-interest with the ultrasound system. In some other examples, processing the Doppler ultrasound data may include isolating the Doppler ultrasound signatures of the vibrating object. These isolated Doppler ultrasound signals may then be overlaid on B-mode images also acquired from the region-of-interest using the ultrasound system.

The determined location of the object may then be presented to a user, or stored for later use or processing, by the computer system, as indicated at step 208. For instance, the locations may be displayed to a user as described above (e.g., by one or more different overlays on B-mode images).

In an example study, vibration-induced shimmering of biopsy markers on color-flow ultrasound was demonstrated in the laboratory using two different media: ultrasound solid gel phantom and firm tofu.

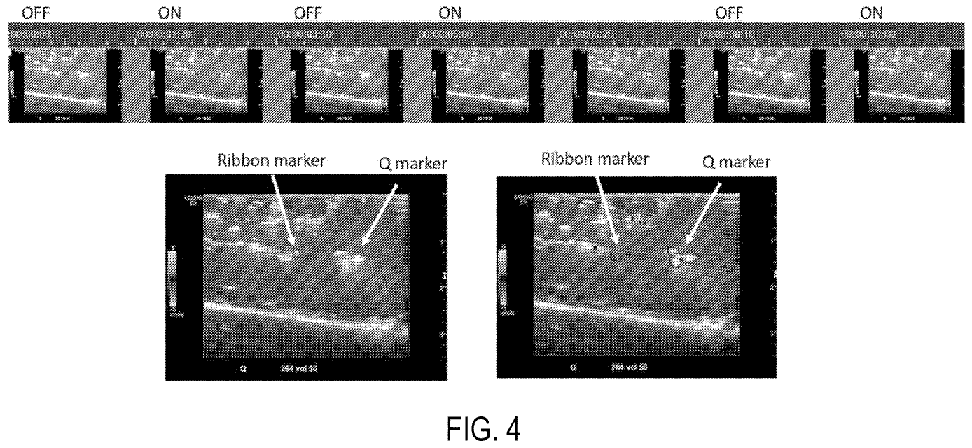

In the first experiment, a Q clip (Tumark® Q, Hologic®, Marlborough, MA, USA) and a ribbon clip (UltraClip™ ribbon, Becton, Dickinson & Co., Franklin Lakes, NJ, USA) were placed in a firm ultrasound phantom gel at approximately 2 cm depth. Using a GE ultrasound scanner (Logiq E9, General Electric Healthcare, Wauwatosa, WI, USA) with a 9-L linear transducer in color-flow Doppler mode, the scale was adjusted from −6 to 6 cm/s, the gain to 0, and the scanning transmit frequency to 6.3 MHz. Periodic vibrations lasting approximately 1 s at 1 s intervals were generated using an actuator embedded in a smart phone placed under the phantom gel. These vibrations were at a frequency of approximately 100 Hz. A periodic shimmering of both the Q clip and ribbon clip were observed, as indicated in FIG. 4. The shimmering (i.e., Doppler ultrasound signature) coincided with the vibrations and disappeared when the vibrations stopped.

In the second experiment, a Q clip and a ribbon clip were placed in a block of firm tofu at 2 cm depth. Using a GE Logiq E9 ultrasound scanner with a 9-L linear probe in color-flow Doppler mode, the scale was adjusted from −6 to 6 cm/s, the gain to 0, and the scanning transmit frequency to 6.3 MHz. A similar experiment was also performed using an L8-18i linear transducer.

The model was placed in a tray on a rubber vibration-dampening pad. The ultrasound probe was aligned with the 2 clips and secured in place with a metallic stabilizer, making sure to keep the 2 clips in view. A small bone conducting actuator was taped to a standard microscopy slide and placed just adjacent to the ultrasound probe. A 100 g weight was also added on top of the actuator/microscopy slide to ensure vibrations are transmitted into the medium.

Continuous vibrations were generated across frequencies ranging from 80 to 2000 Hz while monitoring the resulting changes in the field of vision. The amplitude of vibrations was adjusted as well as the color Doppler gain settings to optimize the signal-to-noise ratio.

A shimmering of both clips was observed when vibration was induced. A frequency between 250 Hz and 275 Hz (e.g., 264 Hz) had favorable visualization conditions, but other settings with good visualization were also identified by adjusting gain and amplitude. The clip had noticeable measured response using color Doppler imaging and pulsed wave Doppler measurement with the sample gate placed over the clip.

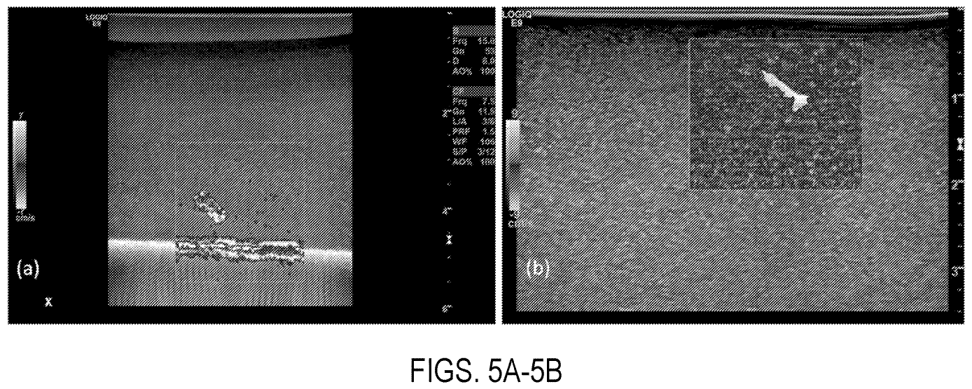

FIGS. 5A and 5B illustrate an example where a biopsy marker is detectable by its positive shimmering feature (FIG. 5A), as well as by a non-shimmering feature relative to background shimmering (FIG. 5B).

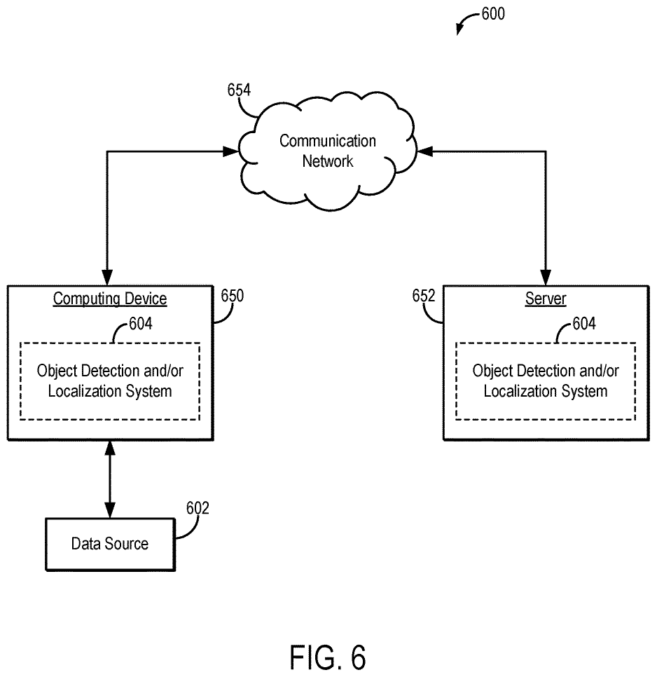

FIG. 6 shows an example of a system 600 for detecting objects, such as biopsy markers, in accordance with some embodiments of the systems and methods described in the present disclosure. As shown in FIG. 6, a computing device 650 can receive one or more types of data (e.g., ultrasound data) from data source 602. In some embodiments, computing device 650 can execute at least a portion of an object detection and localization system 604 to detect, localize, or otherwise visualize the location of an object from data received from the data source 602.

Additionally or alternatively, in some embodiments, the computing device 650 can communicate information about data received from the data source 602 to a server 652 over a communication network 654, which can execute at least a portion of the object detection and localization system 604. In such embodiments, the server 652 can return information to the computing device 650 (and/or any other suitable computing device) indicative of an output of the object detection and localization system 604.

In some embodiments, computing device 650 and/or server 652 can be any suitable computing device or combination of devices, such as a desktop computer, a laptop computer, a smartphone, a tablet computer, a wearable computer, a server computer, a virtual machine being executed by a physical computing device, and so on. The computing device 650 and/or server 652 can also reconstruct images from the data.

In some embodiments, data source 602 can be any suitable source of data (e.g., measurement data, images reconstructed from measurement data, processed image data), such as an ultrasound system, another computing device (e.g., a server storing measurement data, images reconstructed from measurement data, processed image data), and so on. In some embodiments, data source 602 can be local to computing device 650. For example, data source 602 can be incorporated with computing device 650 (e.g., computing device 650 can be configured as part of a device for measuring, recording, estimating, acquiring, or otherwise collecting or storing data). As another example, data source 602 can be connected to computing device 650 by a cable, a direct wireless link, and so on. Additionally or alternatively, in some embodiments, data source 602 can be located locally and/or remotely from computing device 650, and can communicate data to computing device 650 (and/or server 652) via a communication network (e.g., communication network 654).

In some embodiments, communication network 654 can be any suitable communication network or combination of communication networks. For example, communication network 654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 654 can be a local area network, a wide area network, a public network (e.g., the Internet), a private or semi-private network (e.g., a corporate or university intranet), any other suitable type of network, or any suitable combination of networks. Communications links shown in FIG. 6 can each be any suitable communications link or combination of communications links, such as wired links, fiber optic links, Wi-Fi links, Bluetooth links, cellular links, and so on.

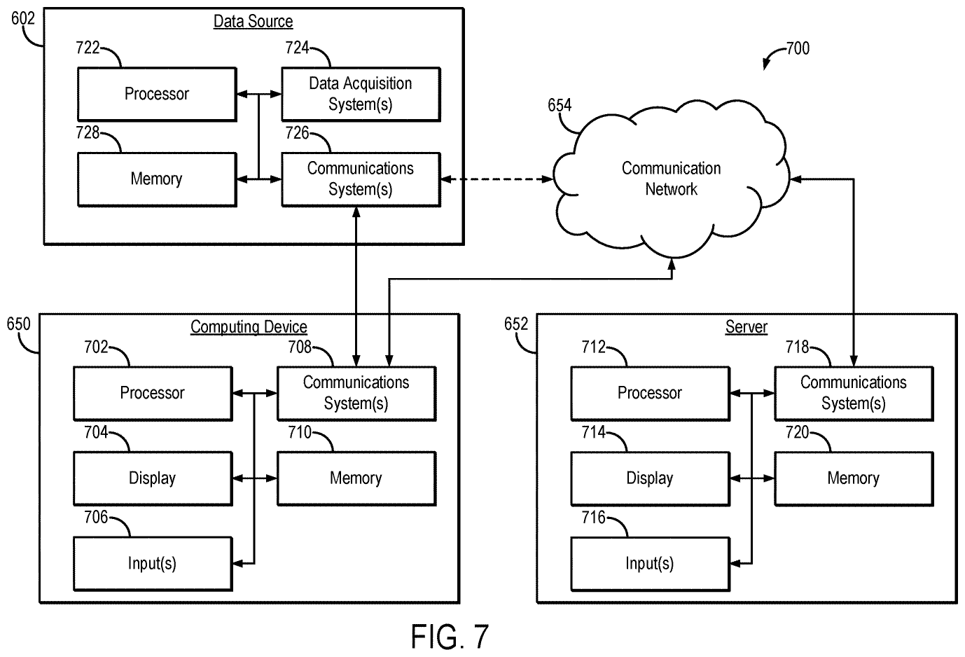

Referring now to FIG. 7, an example of hardware 700 that can be used to implement data source 602, computing device 650, and server 652 in accordance with some embodiments of the systems and methods described in the present disclosure is shown.

As shown in FIG. 7, in some embodiments, computing device 650 can include a processor 702, a display 704, one or more inputs 706, one or more communication systems 708, and/or memory 710. In some embodiments, processor 702 can be any suitable hardware processor or combination of processors, such as a central processing unit (CPU), a graphics processing unit (GPU), and so on. In some embodiments, display 704 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on. In some embodiments, inputs 706 can include any suitable input devices and/or sensors that can be used to receive user input, such as a keyboard, a mouse, a touchscreen, a microphone, and so on.

In some embodiments, communications systems 708 can include any suitable hardware, firmware, and/or software for communicating information over communication network 654 and/or any other suitable communication networks. For example, communications systems 708 can include one or more transceivers, one or more communication chips and/or chip sets, and so on. In a more particular example, communications systems 708 can include hardware, firmware, and/or software that can be used to establish a Wi-Fi connection, a Bluetooth connection, a cellular connection, an Ethernet connection, and so on.

In some embodiments, memory 710 can include any suitable storage device or devices that can be used to store instructions, values, data, or the like, that can be used, for example, by processor 702 to present content using display 704, to communicate with server 652 via communications system(s) 708, and so on. Memory 710 can include any suitable volatile memory, non-volatile memory, storage, or any suitable combination thereof. For example, memory 710 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 710 can have encoded thereon, or otherwise stored therein, a computer program for controlling operation of computing device 650. In such embodiments, processor 702 can execute at least a portion of the computer program to present content (e.g., images, user interfaces, graphics, tables), receive content from server 652, transmit information to server 652, and so on. For example, the processor 702 and the memory 710 can be configured to perform the methods described herein (e.g., the method of FIG. 2).

In some embodiments, server 652 can include a processor 712, a display 714, one or more inputs 716, one or more communications systems 718, and/or memory 720. In some embodiments, processor 712 can be any suitable hardware processor or combination of processors, such as a CPU, a GPU, and so on. In some embodiments, display 714 can include any suitable display devices, such as an LCD screen, LED display, OLED display, electrophoretic display, a computer monitor, a touchscreen, a television, and so on. In some embodiments, inputs 716 can include any suitable input devices and/or sensors that can be used to receive user input, such as a keyboard, a mouse, a touchscreen, a microphone, and so on.

In some embodiments, communications systems 718 can include any suitable hardware, firmware, and/or software for communicating information over communication network 654 and/or any other suitable communication networks. For example, communications systems 718 can include one or more transceivers, one or more communication chips and/or chip sets, and so on. In a more particular example, communications systems 718 can include hardware, firmware, and/or software that can be used to establish a Wi-Fi connection, a Bluetooth connection, a cellular connection, an Ethernet connection, and so on.

In some embodiments, memory 720 can include any suitable storage device or devices that can be used to store instructions, values, data, or the like, that can be used, for example, by processor 712 to present content using display 714, to communicate with one or more computing devices 650, and so on. Memory 720 can include any suitable volatile memory, non-volatile memory, storage, or any suitable combination thereof. For example, memory 720 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 720 can have encoded thereon a server program for controlling operation of server 652. In such embodiments, processor 712 can execute at least a portion of the server program to transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone), and so on.

In some embodiments, the server 652 is configured to perform the methods described in the present disclosure. For example, the processor 712 and memory 720 can be configured to perform the methods described herein (e.g., the method of FIG. 2).

In some embodiments, data source 602 can include a processor 722, one or more data acquisition systems 724, one or more communications systems 726, and/or memory 728. In some embodiments, processor 722 can be any suitable hardware processor or combination of processors, such as a CPU, a GPU, and so on. In some embodiments, the one or more data acquisition systems 724 are generally configured to acquire data, images, or both, and can include an ultrasound system. Additionally or alternatively, in some embodiments, the one or more data acquisition systems 724 can include any suitable hardware, firmware, and/or software for coupling to and/or controlling operations of an ultrasound system. In some embodiments, one or more portions of the data acquisition system(s) 724 can be removable and/or replaceable.

Note that, although not shown, data source 602 can include any suitable inputs and/or outputs. For example, data source 602 can include input devices and/or sensors that can be used to receive user input, such as a keyboard, a mouse, a touchscreen, a microphone, a trackpad, a trackball, and so on. As another example, data source 602 can include any suitable display devices, such as an LCD screen, an LED display, an OLED display, an electrophoretic display, a computer monitor, a touchscreen, a television, etc., one or more speakers, and so on.

In some embodiments, communications systems 726 can include any suitable hardware, firmware, and/or software for communicating information to computing device 650 (and, in some embodiments, over communication network 654 and/or any other suitable communication networks). For example, communications systems 726 can include one or more transceivers, one or more communication chips and/or chip sets, and so on. In a more particular example, communications systems 726 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 728 can include any suitable storage device or devices that can be used to store instructions, values, data, or the like, that can be used, for example, by processor 722 to control the one or more data acquisition systems 724, and/or receive data from the one or more data acquisition systems 724; to generate images from data; present content (e.g., data, images, a user interface) using a display; communicate with one or more computing devices 650; and so on. Memory 728 can include any suitable volatile memory, non-volatile memory, storage, or any suitable combination thereof. For example, memory 728 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 728 can have encoded thereon, or otherwise stored therein, a program for controlling operation of data source 602. In such embodiments, processor 722 can execute at least a portion of the program to generate images, transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

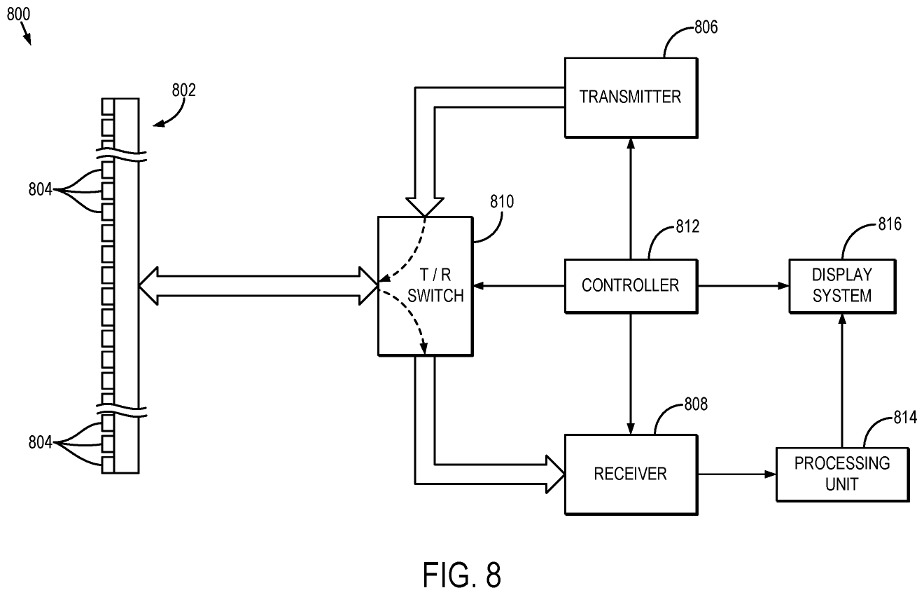

FIG. 8 illustrates an example of an ultrasound system 800 that can implement the methods described in the present disclosure. The ultrasound system 800 includes a transducer array 802 that includes a plurality of separately driven transducer elements 804. The transducer array 802 can include any suitable ultrasound transducer array, including linear arrays, curved arrays, phased arrays, and so on. Similarly, the transducer array 802 can include a 1D transducer, a 1.5D transducer, a 1.75D transducer, a 2D transducer, a 3D transducer, and so on.

When energized by a transmitter 806, a given transducer element 804 produces a burst of ultrasonic energy. The ultrasonic energy reflected back to the transducer array 802 (e.g., an echo) from the object or subject under study is converted to an electrical signal (e.g., an echo signal) by each transducer element 804 and can be applied separately to a receiver 808 through a set of switches 810. The transmitter 806, receiver 808, and switches 810 are operated under the control of a controller 812, which may include one or more processors. As one example, the controller 812 can include a computer system.

The transmitter 806 can be programmed to transmit unfocused or focused ultrasound waves. In some configurations, the transmitter 806 can also be programmed to transmit diverged waves, spherical waves, cylindrical waves, plane waves, or combinations thereof. Furthermore, the transmitter 806 can be programmed to transmit spatially or temporally encoded pulses.

The receiver 808 can be programmed to implement a suitable detection sequence for the imaging task at hand. In some embodiments, the detection sequence can include one or more of line-by-line scanning, compounding plane wave imaging, synthetic aperture imaging, and compounding diverging beam imaging.

In some configurations, the transmitter 806 and the receiver 808 can be programmed to implement a high frame rate. For instance, a frame rate associated with an acquisition pulse repetition frequency (PRF) of at least 100 Hz can be implemented. In some configurations, the ultrasound system 800 can sample and store at least one hundred ensembles of echo signals in the temporal direction.

The controller 812 can be programmed to implement or otherwise design an imaging sequence using the techniques described in the present disclosure, or as otherwise known in the art. In some embodiments, the controller 812 receives user inputs defining various factors used in the design of the imaging sequence. As described above, the imaging sequence may include a detection mode that is optimized to measure Doppler ultrasound signatures generated by a vibrating object. Additionally or alternatively, the imaging sequence may include vibration stimulus parameter settings (e.g., vibration amplitude, vibration frequency, vibration onset times, vibration durations, and so on) for controlling the external vibrational device.

A scan can be performed by setting the switches 810 to their transmit position, thereby directing the transmitter 806 to be turned on momentarily to energize transducer elements 804 during a single transmission event according to the selected imaging sequence. The switches 810 can then be set to their receive position and the subsequent echo signals produced by the transducer elements 804 in response to one or more detected echoes are measured and applied to the receiver 808. The separate echo signals from the transducer elements 804 can be combined in the receiver 808 to produce a single echo signal.

The echo signals are communicated to a processing unit 814, which may be implemented by a hardware processor and memory, to process echo signals or images generated from echo signals. As an example, the processing unit 814 can process ultrasound data to determine the location of an object being vibrated by mechanical vibrations generated by an external vibrational device using the methods described in the present disclosure. Images produced from the echo signals by the processing unit 814 can be displayed on a display system 816.

The present disclosure has described one or more preferred embodiments, and it should be appreciated that many equivalents, alternatives, variations, and modifications, aside from those expressly stated, are possible and within the scope of the invention.

1. A method for determining a location of an object in a region-of-interest in a subject using an ultrasound system, the method comprising:
(a) generating mechanical vibrations in a region-of-interest containing an object in a subject using an external vibrational device;
(b) acquiring ultrasound data from the region-of-interest using an ultrasound system while the mechanical vibrations are propagating within the region-of-interest such that the object vibrates in response thereto;
(c) processing the ultrasound data with a computer system to determine a location of the object within the region-of-interest based on Doppler ultrasound signatures generated by vibrations of the object interacting with ultrasound waves incident on the object; and
(d) outputting the location of the object via the computer system.

2. The method of claim 1, wherein the mechanical vibrations are generated using the external vibrational device while the external vibrational device is coupled to a skin surface of the subject.

3. The method of claim 1, wherein the vibrational device comprises a mechanical actuator.

4. The method of claim 1, wherein the ultrasound data are acquired by operating the ultrasound system in a Doppler imaging mode.

5. The method of claim 4, wherein the Doppler imaging mode comprises a color-flow mode.

6. The method of claim 4, wherein the Doppler imaging mode comprises a power Doppler imaging mode.

7. The method of claim 1, wherein processing the ultrasound data comprises isolating the Doppler ultrasound signatures from the ultrasound data.

8. The method of claim 1, wherein presenting the location of the object to a user via the computer system comprises overlaying the processed ultrasound data with B-mode images acquired from the region-of-interest with the ultrasound system.

9. The method of claim 1, wherein the mechanical vibrations are generated as periodic vibrations each having a duration and each separated in time by an interval.

10. The method of claim 9, wherein the duration is 1 second.

11. The method of claim 10, wherein the interval is 1 second.

12. The method of claim 9, wherein the mechanical vibrations are generated with a frequency selected from a range of 80-2000 Hz.

13. The method of claim 1, wherein the mechanical vibrations are generated as continuous vibrations.

14. The method of claim 13, wherein the mechanical vibrations are generated with a frequency selected from a range of 80 to 2000 Hz.

15. The method of claim 14, wherein the mechanical vibrations are generated with a frequency selected from a range of 250 to 275 Hz.

16. The method of claim 15, wherein the mechanical vibrations are generated with a frequency of 264 Hz.

17. The method of claim 1, wherein the mechanical vibrations are generated by driving the external vibration device using white noise as an input signal.

18. The method of claim 1, wherein the computer system receives pressure measurements from a pressure gauge coupled to an ultrasound transducer of the ultrasound system and processes the pressure measurements to provide feedback for maintaining an optimal pressure against the subject.

19. The method of claim 1, wherein a second object is arranged in the subject and vibrations of the object interact with the second object to cause the second object to vibrate, such that a location of the second object can be determined from the ultrasound data based on second Doppler ultrasound signatures associated with the second object.

20. The method of claim 1, wherein the vibrational device is coupled to an ultrasound transducer of the ultrasound system.

21. The method of claim 1, wherein the Doppler ultrasound signatures are indicative of a positive shimmering of the object relative to surrounding tissues in the region-of-interest.

22. The method of claim 1, wherein the Doppler ultrasound signatures are indicative of a negative shimmering of the object relative to surrounding tissues in the region-of-interest.

23. The method of claim 1, wherein microbubbles associated with the object are elicited to generate a Doppler shimmering signature in response to the mechanical vibrations generated by the external vibrational device.

24. The method of claim 1, wherein the external vibrational device comprises a plurality of actuators and the mechanical vibrations are generated using one or more of the plurality of actuators.

25. The method of claim 24, wherein the plurality of actuators are arranged in a ring.
